(19) United States
(12) Patent Application Publication
(10) Pub. No.: US 2003/0203044 A1
(43) Pub. Date: Oct. 30, 2003

(54) Wound-treating gel

(76) Inventor: Anthony J. Moravec, Columbus, IN (US)

Correspondence Address: Todd T. Taylor, TAYLOR & AUST, P.C., 142 S. Main St., P.O. Box 560, Avilla, IN, 46710, US

(21) Appl. No.: 10/422,161

(22) Filed: Apr. 24, 2003

Related U.S. Application Data
(60) Provisional application No. 60/375,651, filed on Apr. 26, 2002.

(51) Int. Cl. A61K 9/70; A61K 33/42; B65B 55/02

(52) U.S. Cl. 424/601; 53/425

(57) ABSTRACT

A wound-treating gel is provided from an inorganic salt. The gel can be packed in a multi-dose container, such as a bag-in-can dispensing system, and is sterilized by gamma irradiation.

### CROSS-REFERENCE TO RELATED APPLICATIONS

The present application claims benefit of United States Provisional Application No, 60/375,651, filed Apr. 26, 2002.

### FIELD OF THE INVENTION

The present invention pertains generally to wound dressings, and, more particularly, the present invention pertains to the formulation, production, packaging and sterilizing of hydrating agents, or liquid bandages, used to promote healing of wounds of many types.

### BACKGROUND OF THE INVENTION

As used herein, the term “wound” shall be known to include, but not be limited to various types of cuts, abrasions, burns including sunburn, surgical incisions, pressure ulcers, diabetic ulcers and other injuries and maladies, both chronic and non-chronic. It is known that, when treating such wounds, the healing rate can be improved by controlling the environment around the wound during the healing process. Many wounds such as those listed above heal faster with the application of hydrating agents, as compared to the simple application of bandages. Various hydrating agents have been used for the treatment of wounds, with varying degrees of success.

Wound-treating formulations in the nature of gels provide certain advantages in both handling and use. The application of a gel is often easier than the application of other, more liquid, types of wound-treating formulations. A gel can be applied accurately to the wound, providing a continuous, uninterrupted covering over the wound. The entire wound is thereby subjected to the improved healing environment created by the gel, and the wound can heal evenly and consistently throughout. Gels can be made to stay where applied, providing prolonged control of the healing environment.

Wound treatment formulations should be sterile, to prevent contamination and infection of the wound. Known sterilizing techniques include heating, filtration and gamma irradiation. Filtration does not wok well for sterilizing gels, because the thickness of the gel makes filtration difficult and impractical. Known gels for the treatment of wounds are organic based gels. Gamma radiation breaks down the structure of organic gels, making the gel more liquid. Therefore, wound gels have been sterilized with heat.

To package a multi-dose quantity of any sterile substance, a container holding the substance advantageously will maintain the sterility of the undispensed portion of substance. Hermetically sealed, pressurized barrier cans sterilized by gamma irradiation have been known for this purpose. However, wound-treating gels have not been packaged in this manner in that, as explained above, heating has been the only suitable sterilization technique for known wound treating gels. Heating a gel or other substance in a pressurized can sufficiently to sterilize the substance can disrupt the seals in the can, destroying the sterile-maintaining capabilities of the can, or the pressurization of the can for dispensing the substance.

Consequently, wound treating gels commonly have been packaged in only single dose quantities, in a variety of containers or packages that can withstand heating to sterilize the gel. While sometimes useful, single dose packages of wound treating gels also have disadvantages. Opening the sleeves or other packages containing the gel is often difficult. The package may contain more gel than is necessary for a small wound, and the remaining unused gel is wasted in that the sterility thereof can not be ensured. For large wounds, several packages of gel may be required, slowing the application as the packages are opened, and increasing the amount of material to be disposed. Packaging, storing and handling small, single dose quantities of wound treating gels are expensive and inconvenient.

Small, non-barrier packaging also has been used. Upon opening the package, the gel is exposed to potential contamination. To maintain the sterility of the gel, prolonging its “opened” shelf-life, it is known to include preservatives in the gel, to inhibit the growth of contaminants. An undesirable side effect from a preservative in a wound treating gel is that the preservative also may inhibit initial stages of tissue growth during the healing process.

What is needed is a wound treating gel that can be sterilized and stored in, and dispensed from a multi-dose quantity of the gel, and that does not require the inclusion of a preservative.

### SUMMARY OF THE INVENTION

The present invention provides a wound treating gel that can be sterilized by irradiation and packed in a multi-dose dispensing canister.

In one form thereof, the present invention provides a method of packaging a gel for treating wounds having steps of providing a wound-treating gel and a package for the gel of sufficient size to hold a multi-dose quantity of the gel; placing a multi-dose quantity of the gel in the package; and irradiating the package with the multi-dose volume of gel therein sufficiently to sterilize the gel.

In another form thereof, the present invention provides a method of making a wound-treating gel comprising steps of providing a gel made from an inorganic salt and water; packaging the gel and irradiating the packaged gel sufficiently to sterilize the gel.

In still another form thereof, the present invention provides a. wound-treating gel comprising a sterilized mixture of water in an amount of 92.3 percent of the mixture, by weight; sodium magnesium silicate in an amount of about 7.0 percent of the mixture, by weight and monobasic sodium phosphate in an amount of about 0.7 percent of the mixture, by weight.

In a further form thereof, the present invention provides a wound-treating gel comprising a mixture of water, sodium magnesium silicate and monobasic sodium phosphate.

An advantage of the present invention is providing a wound-treating gel that can be sterilized and packaged in a multi-dose quantity.

Another advantage of the present invention is providing an inorganic wound-treating gel.

A further advantage of the present invention is providing a wound-treating gel that can be sterilized by gamma radiation.

A still further advantage of the present invention is providing a wound-treating gel that can be packaged in and dispensed from a pressurized can.

Still another advantage of the present invention is providing a wound-treating gel that contains no preservatives, and improves healing as compared to known wound-treating gels.

Other features and advantages of the invention will become apparent to those skilled in the art upon review of the following detailed description and claims.

Before the embodiments of the invention are explained in detail, it is to be understood that the invention is not limited in its application to the details of construction and the arrangements of the components set forth in the following description. The invention is capable of other embodiments and of being practiced or being carried out in various ways. Also, it is to be understood that the phraseology and terminology used herein are for the purpose of description and should not be regarded as limiting. The use herein of “including” and “comprising” and variations thereof is meant to encompass the items listed thereafter and equivalents thereof, as well as additional items and equivalents thereof.

### DETAILED DESCRIPTION OF THE PREFERRED EMBODIMENT

The present wound-treating gel is a sterile gel composed of water, magnesium silicate and monobasic sodium phosphate that can be packaged in a pressurized bag-in-can dispensing system. The present wound-treating gel is indicated for use as a hydrating agent to promote a moist wound environment for optimal wound healing. It is indicated for use on cuts, abrasions, first and second degree burns including sunburn, post-surgical incisions, pressure ulcers, diabetic ulcers and other similar injuries and maladies, both chronic and non-chronic, which are referred to herein individually and collectively as “wound” or “wounds”.

The present invention takes advantages of the discovery that inorganic gels can be sterilized by radiation, without degrading the gel composition. Unlike organic gels that liquefy when irradiated, inorganic gels remain as gels. Thus, a wound treating gel can be made from an inorganic salt, packaged in a large volume, and sterilized by irradiation after packaging. A suitable composition for a wound-treating gel according to the present invention is made of the following:

| Ingredient | Quantity | % of W/W |
|---|---|---|
| Purified Water, USP (1) | 1571.50 Kg. | 89.80% |
| Purified Water, USP (2) | 43.75 Kg. | 2.50% |
| Optigel SH | 122.50 Kg. | 7.00% |
| (sodium magnesium silicate) | | |
| Monobasic Sodium Phosphate | 12.25 Kg. | 0.70% |
| USP (Monohydrate) | | |

The wound-treating gel is made by adding the purified water USP (1) to a mixer, such as a Lee Tri-mix, having an agitator and a disperser. The agitator is rotated at about 5 rpm, and the disperser is rotated at about 500 rpm. The sodium magnesium silicate (available under the name Optigel SH from Sud Chemie) is sifted slowly into the mixer, and mixing continues at least about two hours to ensure thorough mixing and incorporation of the Optigel SH. In a separate container or mixer, such as a Lightnin mixer, the purified water USP (2) is mixed with the monobasic sodium phosphate (monohydrate) until the monobasic sodium phosphate is completely dissolved. This later solution is then combined with the Optigel SH mixture, and further mixed until homogenous. The completed gel is a colorless, odorless, viscous translucent gel, of between about 6.5 pH and about 8.5 pH.

Wound-treating gel of the present invention can be sterilized by gamma irradiation, and therefore can be packaged in a multi-dose container from which required quantities can be dispensed while retaining the undispensed portion in a sterile environment. An aerosol bag-in-can dispensing system is suitable. Bag-in-can dispensing systems are known for use in dispensing other sterile compositions, but have not been used for dispensing sterile gels, because of the aforedescribed difficulties inherent in sterilizing known organic gels in large volumes and within sealed, pressurized containers. A barrier type package is desirable in that, unlike a tube or other similar container, a bag-in-can container is never “opened”. The content of the can is never exposed to an environment outside of the can until it is dispensed from the can for use. Thus, so-called “after opening” expiration dating is not required. Since the wound-treating gel in the can is retained in a sterile environment, it is not necessary to include preservatives in the gel. Preservatives are commonly added to known wound-treating gels packaged in tubes or the like, to prolong the life of the product after the tube is opened. In addition to killing unwanted bacteria after the gel is exposed to the external environment, preservatives can have the undesirable side effect of killing growing skin cells, thereby slowing the healing process. By eliminating the need for preservatives, wound-treating gel of the present invention creates an environment over the wound that promotes faster healing than does wound-treating gel having a preservative therein.

The completed gel of the present invention is packaged in a bag-in-can container in a multi-dose quantity. By way of example, both 1.5 ounce and 6.0 ounce bag-in-can dispensing systems can be used. During filling, the bag is inspected and placed in the can. The can is crimped closed, and charged with compressed air. The finished gel is pressure filled into the bag in the can, and the dispensing structure is finished by placement of the actuator on the can. An overcap is placed on the can. Thereafter, multiple cans can be accumulated into packs, such as by shrink wrapping, and multiple packs placed into a shipping container, such as a box.

The packaged gel is sterilized using ⁶⁰Cobalt gamma irradiation, consistent with the procedures and requirements of ISO 11137, “Sterilization Of Health Care Products—Requirements For Validation And Routine Control—Radiation Sterilization”, to assure a sterilization assurance level of 10⁻⁶. Such sterilization techniques, using gamma irradiation, are known for sterilizing other materials, such as saline, but have not been used for sterilizing gels because of the previously mentioned problems associated with irradiation of organic gels. While the gel can be sterilized in cans individually, advantageously packs of multiple cans, or containers such as boxes having multiple packs of multiple cans can be sterilized simultaneously by irradiation. Such sterilization techniques for multiple packs or multiple containers are well known to those skilled in the art.

Bio-compatibility of the wound-treating gel has been demonstrated through human clinical studies. Lasers were used to create three superficial (epidermal) wounds on volunteers. Each wound was treated with one of three wound-treating products in the study, one of which was the present wound-treating gel, and the others of which were known, commercially available organic gel based products. Clinical scores were recorded at days 4, 6, 8 and 10 during healing, and revealed that the present wound-treating gel performed statistically as effective as known organic gels on measures for edema, epithelialization, erythema or scabbing. The present wound-treating gel was found to be statistically superior to the known products for wound healing as judged by transepidermal water loss score.

In the use of wound-treating gel made in accordance with the present invention, a wound to be treated is cleaned before application of the present wound-treating gel. The gel can be applied directly to the wound site from the bag-in-can dispensing system, in a quantity sufficient to form a continuous covering over the wound. The thickness of the layer of wound-treating gel should be from about one-eighth to about one-quarter inch thick. The covering should be changed as necessary to maintain a continuous, moist covering over the wound. External bandaging also can be used to cover the layer of wound-treating gel.

The present invention provides an easily handled, sterilized gel for the treatment of wounds. Since the gel can be sterilized with radiation, large quantities thereof can be packaged and sterilized conveniently. Compared to known, single application packs, or known multi-dose packs having limited shelf-life after opening, waste is reduced with the present gel in that only the required volume of gel is dispensed from the container, and the remaining gel is held in a sterile environment. By eliminating the need for preservatives, the present gel creates an over-the-wound environment more conducive to rapid healing.

Variations and modifications of the foregoing are within the scope of the present invention. It is understood that the invention disclosed and defined herein extends to all alternative combinations of two or more of the individual features mentioned or evident from the text. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

Various features of the invention are set forth in the following claims.

What is claimed is:

1. A method of packaging a gel for treating wounds, said method comprising steps of:
providing a wound-treating gel and a package for the gel of sufficient size to hold a multi-dose quantity of the gel;
placing a multi-dose quantity of the gel in the package; and
irradiating the package with the multi-dose volume of gel therein sufficiently to sterilize the gel.

2. The method of claim 1, said irradiating step performed with ⁶⁰Cobalt gamma radiation.

3. The method of claim 1, said providing step including providing a bag-in-can pressurized dispensing system, and said placing step performed by placing a multi-dose quantity of the gel in the can.

4. The method of claim 3, said irradiating step performed with ⁶⁰Cobalt gamma radiation.

5. The method of claim 1, said providing step including providing a wound-treating gel of sodium magnesium silicate.

6. The method of claim 5, said irradiating step performed with ⁶⁰Cobalt gamma radiation.

7. The method of claim 1, said providing step including providing an inorganic wound-treating gel.

8. The method of claim 7, said irradiating step performed with ⁶⁰Cobalt gamma radiation.

9. The method of claim 7, said providing step including providing a bag-in-can pressurized dispensing system, and said placing step performed by placing a multi-dose quantity of the inorganic wound-treating gel in the can.

10. The method of claim 9, said radiating step performed with ⁶⁰Cobalt gamma radiation.

11. A method of making a wound-treating gel comprising steps of:
providing a gel made from an inorganic salt and water;
packaging the gel; and
irradiating the packaged gel sufficiently to sterilize the gel.

12. The method of claim 11, said irradiating step performed with ⁶⁰Cobalt gamma radiation.

13. The method of claim 11, said step of providing a gel including mixing sodium magnesium silicate, water and monobasic sodium phosphate.

14. The method of claim 13, said irradiating step performed with ⁶⁰Cobalt gamma radiation.

15. The method of claim 11, said step of providing a gel including creating a first mixture for the gel by mixing by weight about 89.8 percent purified water with about 7.0 percent sodium magnesium silicate; creating a second mixture for said gel by mixing by weight about 2.5 percent purified water with about 0.7 percent monobasic sodium phosphate; and thereafter mixing together the first and second mixtures.

16. The method of claim 11, said step of providing a gel performed by separately mixing a first portion of water with sodium magnesium silicate and mixing a second portion of water with monobasic sodium phosphate, and thereafter combing the two mixtures.

17. The method of claim 16, said irradiating step performed with ⁶⁰Cobalt gamma radiation.

18. A wound-treating gel comprising a sterilized mixture of:
water in an amount of 92.3 percent of said mixture, by weight;
sodium magnesium silicate in an amount of about 7.0 percent of said mixture, by weight; and
monobasic sodium phosphate in an amount of about 0.7 percent of said mixture, by weight.

19. A wound-treating gel comprising a sterilized mixture of:
water;
sodium magnesium silicate; and
monobasic sodium phosphate.

20. The wound-treating gel of claim 19, said mixture being sterilized by ⁶⁰Cobalt gamma radiation.
